(19) United States
(12) Patent Application Publication
(10) Pub. No.: US 2004/0023858 A1
(43) Pub. Date: Feb. 5, 2004

(54) Antifungal combination therapy

(76) Inventors: Fumiaki Ikeda, Osaka (JP); Etsuko Watabe, Osaka (JP); Satoru Matsumoto, Osaka (JP); Tomoe Ushitani, Osaka (JP); Yasuto Koide, Osaka (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/344,008

(22) PCT Filed: Aug. 13, 2001
(86) PCT No.: PCT/JP01/07009

(30) Foreign Application Priority Data
Aug. 14, 2000 (AU) PQ9387

(51) Int. Cl. A61K 38/12

(52) U.S. Cl. 514/9

(57) ABSTRACT

There is described antifungal combination use of Granulocyte-colony stimulating factor with a lipopeptide compound (I) as described herein.

### TECHNICAL FIELD

The present invention relates to antifungal combination use of Granulocyte-colony stimulating factor (G-CSF) with a lipopeptide compound antifungal agent.

### BACKGROUND ART

There is an increasing need for agents which are effective against opportunistic mycotic infections by such agents as Cryptococcus, Candida, Aspergillus, Histoplasma, Coccidioides, Paracoccidioides, Blastonyces, Fusarium, Sporothrix, Trichosporon, Rhizopus, Pseudallescheria, dermatophytes, Paeciliomyces, Alternaria, Curvularia, Exophiala, Wangiella, Penicillium, Saccharomyces, Dematiaceous fungi, pneumocystis carinii and so on. The lipopeptide compound [I] is cyclic hexapeptide which inhibits cell wall 1,3β-D-glucan synthesis. The lipopeptide compound [I] has shown potent in vivo activity against Candida, Pneumocystis carinii, Aspergillus, as well as the other fungal pathogens listed above (U.S. Pat. Nos. 5,502,033, 5,376,634, 5,569,646, W096/11210 and W099/40108).

### DISCLOSURE OF THE INVENTION

The present invention relates to antifungal combination use of Granulocyte-colony stimulating factor (G-CSF) with a lipopeptide compound antifungal agent. More particularly, the present invention relates to antifungal combination use of G-CSF with a lipopeptide compound [I] of the following formula:

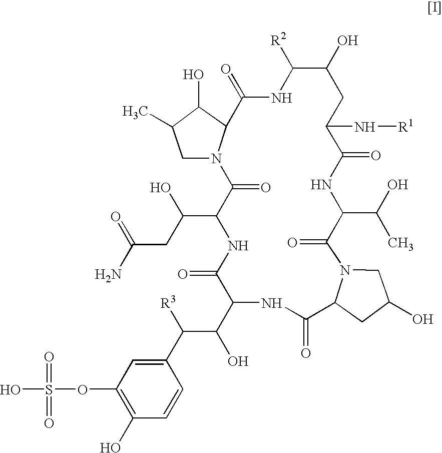

Wherein R¹ is acyl group,

R² is hydrogen or hydroxy and

R³ is hydrogen or hydroxy,

or a salt thereof.

Suitable salt of the lipopeptide compound [I] is a pharmaceutically acceptable and conventional non-toxic salt, and may include a salt with a base or an acid addition salt such as a salt with an inorganic base, for example, an alkali metal salt (e.g., sodium salt, potassium salt, etc.), an alkaline earth metal salt (e.g., calcium salt, magnesium salt, etc.), an ammonium salt;

a salt with an organic base, for example, an organic amine salt (e.g., triethylamine salt, diisopropylethylamine salt, pyridine salt, picoline salt, ethanolamine salt, triethanolamine salt, dicyclohexylamine salt, N,N′-dibenzylethylenediamine salt, etc.);

an inorganic acid addition salt (e.g., hydrochloride hydrobromide, sulfate, phosphate, etc.);

an organic carboxylic sulfonic acid addition salt (e.g., formate, acetate, trifluoroacetate, maleate, tartrate, fumarate, methanesulfonate, benzenesulfonate, toluenesulfonate, etc.);

a salt with a basic or acidic amino acid (e.g., arginine, aspartic acid, glutamic acid, etc.).

It is to be noted that each of the lipopeptide compound [I] may include one or more stereoisomer(s) such as optical isomer(s) and geometrical isomer(s) due to asymmetric carbon atom(s) and double bond(s), and all such isomer(s) and the mixture thereof are included within the scope of the present invention.

The lipopeptide compound [I] or a salt thereof includes solvated compound [e.g., enclosure compound (e.g., hydrate, etc.)].

The lipopeptide compound [I] or a salt thereof includes both its crystal form and non-crystal form.

It should be understood that the lipopeptide compound [I] in the present invention may include the prodrug form.

Suitable example of “acyl group” may include aliphatic acyl, aromatic acyl, arylaliphatic acyl and heterocyclic-aliphatic acyl derived from carboxylic acid, carbonic acid, carbamic acid, sulfonic acid, and the like.

Suitable example of said “acyl group” may be illustrated as follows.

Aliphatic acyl such as lower or higher alkanoyl (e.g., formyl, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl, icosanoyl, etc.);

lower or higher alkoxycarbonyl (e.g., methoxycarbonyl, ethoxycarbonyl, t-butoxycarbonyl, t-pentyloxycarbonyl, heptyloxycarbonyl, etc.);

lower or higher alkylsulfonyl (e.g., methylsulfonyl, ethylsulfonyl, etc.);

lower or higher alkoxysulfonyl (e.g., methoxysulfonyl, ethoxysulfonyl, etc.); or the like;

Aromatic acyl such as aroyl (e.g., benzoyl, toluoyl, naphthoyl, etc.); aroyl which has one or more suitable substituent(s);

ar(lower)alkanoyl [e.g., phenyl(C₁-C₆)alkanoyl (e.g., phenylacetyl, phenylpropanoyl, phenylbutanoyl, phenylisobutanoyl, phenylpentanoyl, phenylhexanoyl, etc.), naphthyl(C₁-C₆)alkenoyl (e.g., naphthylacetyl, naphthylpropenoyl, naphthylbutanoyl, etc.), etc.];

ar(lower)alkenoyl [e.g., phenyl(C₃-C₆)alkenoyl (e.g., phenylpropenoyl, phenylbutenoyl, phenylmethacryloyl, phenylpentanoyl, phenylhexenoyl, etc.), naphthyl(C₃-C₆)alkenoyl (e.g., naphthylpropenoyl, naphthylbutenoyl, etc.), etc.];

ar(lower)alkoxycarbonyl [e.g., phenyl(C₁-C₆)alkoxycarbonyl (e.g., benzyloxycarbonyl, etc.), fluorenyl(C₁-C₆)alkoxy-carbonyl (e.g., fluorenylmethyloxycarbonyl, etc.), etc.];

aryloxycarbonyl (e.g., phenoxycarbonyl, naphthyloxycarbonyl, etc.);

aryloxy(lower)alkanoyl (e.g., phenoxyacetyl, phenoxypropionyl, etc.);

arylcarbamoyl (e.g., phenylcarbamoyl, etc.);

arylthiocarbamoyl (e.g., phenylthiocarbamoyl, etc.);

arylglyoxyloyl (e.g., phenylglyoxyloyl, naphthylglyoxyloyl, etc.);

arylsulfonyl which may have 1 to 4 lower alkyl (e.g., phenylsulfonyl, p-tolylsulfonyl, etc.); or the like;

Heterocyclic aryl such as heterocycliccarbonyl; heterocyclic(lower)alkanoyl (e.g., heterocyclicacetyl, heterocyclicpropanoyl, heterocyclicbutanoyl, heterocyclicpentanoyl, heterocyclichexanoyl, etc.);

heterocyclic(lower)alkenoyl (e.g., heterocyclicpropenoyl, heterocyclicbutenoyl, heterocyclicpentenoyl, heterocyclichexenoyl, etc.);

heterocyclicglyoxyloyl; or the like.

Among them, more preferred “acyl group” is aroyl which has one or more suitable substituent(s).

Suitable example of “suitable substituent(s)” in the term of “aroyl which has one or more suitable substituent(s)” may be heterocyclic group substituted with aryl having lower alkoxy, heterocyclic group substituted with aryl having lower alkoxy(lower)alkoxy, heterocyclic group substituted with aryl having lower alkoxy(higher)alkoxy, heterocyclic group substituted with aryl having cyclo(lower)alkyloxy, heterocyclic group substituted with aryl having heterocyclic group, heterocyclic group substituted with cyclo(lower)alkyl having cyclo(lower)alkyl, heterocyclic group substituted with aryl having aryl substituted with lower alkoxy(lower)alkoxy, heterocyclic group substituted with aryl having heterocyclic group substituted with cyclo(lower)alkyl;

in which the preferred one may be unsaturated 3 to 8-membered heteromonocyclic group containing 1 to 2 oxygen atom(s) and 1 to 3 nitrogen atom(s) substituted with phenyl having (C₄-C₆)alkoxy, unsaturated condensed heterocyclic group containing 1 or 2 sulfur atom(s) and 1 to 3 nitrogen atom(s) substituted with phenyl having (C₄-C₆)alkoxy, unsaturated 3 to 8-membered heteromonocyclic group containing 1 or 2 sulfur atom(s) and 1 to 3 nitrogen atom(s) substituted with phenyl having (C₁-C₄)alkoxy-(C₄-C₆)alkoxy, unsaturated 3 to 8-membered heteromonocyclic group containing 1 or 2 sulfur atom(s) and 1 to 3 nitrogen atom(s) substituted with phenyl having (C₁-C₄)alkoxy(C₇-C₁₄)alkoxy, saturated 3 to 8-membered heteromonocyclic group containing 1 to 4 nitrogen atom(s) substituted with phenyl having (C₁-C₄)alkoxy(C₇-C₁₄)alkoxy, unsaturated condensed heterocyclic group containing 1 or 2 sulfur atom(s) and 1 to 3 nitrogen atom(s) substituted with phenyl having cyclo(C₄-C₆)alkyloxy, unsaturated condensed heterocyclic group containing 1 or 2 sulfur atom(s) and 1 to 3 nitrogen atom(s) substituted with phenyl saturated 3 to 8-membered heteromonocyclic group containing 1 or 2 oxygen atom(s) and 1 to 3 nitrogen atom(s), saturated 3 to 8-membered heteromonocyclic group containing 1 to 4 nitrogen atom(s) substituted with cyclo(C₄-C₆)alkyl having cyclo(C₄-C₆)alkyl, unsaturated 3 to 8-membered heteromonocyclic group containing 1 or 2 sulfur atom(s) and 1 to 3 nitrogen atom(s) substituted with phenyl having phenyl substituted with (C₁-C₄)alkoxy(C₁-C₄)alkoxy, unsaturated 3 to 8-membered heteromonocyclic group containing 1 or 2 sulfur atom(s) and 1 to 3 nitrogen atom(s) substituted with phenyl having saturated 3 to 8-membered heteromonocyclic group containing 1 to 4 nitrogen atom(s) substituted with cyclo(C₄-C₆)alkyl, unsaturated condensed heterocyclic group containing 1 or 2 sulfur atom(s) and 1 to 3 nitrogen atom(s) substituted with phenyl having saturated 3 to 8-membered heteromonocyclic group containing 1 to 4 nitrogen atom(s) having cyclo(C₄-C₆)alkyl, etc.

Among them, the most preferred one may be isoxazolyl substituted with phenyl having pentyloxy, imidazothiadiazolyl substituted with phenyl having pentyloxy, thiadiazolyl substituted with phenyl having methoxyhexyloxy, thiadiazolyl substituted with phenyl having methoxyoctyloxy, thiadiazolyl substituted with phenyl having methoxyheptyloxy, imidazothiadiazolyl substituted with phenyl having cyclohexyloxy, imidazothiadiazolyl substituted with phenyl having dimethylmorpholino, piperazinyl substituted with phenyl having methoxyheptyloxy, piperazinyl substituted with phenyl having methoxyoctyloxy, piperazinyl substituted with cyclohexyl having cyclohexyl, thiadiazolyl substituted with phenyl having phenyl substituted with methoxyethoxy, thiadiazolyl substituted with phenyl having phenyl substituted with methoxybutoxy, thiadiazolyl substituted with phenyl having phenyl substituted with ethoxypropoxy, imidazothiadiazolyl substituted with phenyl having piperazinyl substituted with cyclohexyl, imidazothiadiazolyl substituted with phenyl having piperazinyl substituted with cyclohexyl.

The more suitable example of “acyl group” of R¹ may be benzoyl which has isoxazolyl substituted with phenyl having pentyloxy, benzoyl which has imidazolthiadiazolyl substituted with phenyl having pentyloxy, benzoyl which has thiadiazolyl substituted with phenyl having methoxyhexyloxy, benzoyl which has thiadiazolyl substituted with phenyl having methoxyoctyloxy, benzoyl which has thiadiazolyl substituted with phenyl having methoxyheptyloxy, benzoyl which has imidazothiadiazolyl substituted with phenyl having cyclohexyloxy, benzoyl which has imidazothiadiazolyl substituted with phenyl having dimethylmorpholino, benzoyl which has piperazinyl substituted with phenyl having methoxyheptyloxy, benzoyl which has piperazinyl substituted with phenyl having methoxyoctyloxy, benzoyl which has piperazinyl substituted with cyclohexyl having cyclohexyl, benzoyl which has thiadiazolyl substituted with phenyl having phenyl substituted with methoxyethoxy, benzoyl which has thiadiazolyl substituted with phenyl having phenyl substituted with methoxybutoxy, benzoyl which has thiadiazolyl substituted with phenyl having phenyl substituted with ethoxypropoxy, benzoyl which has imidazothiadiazolyl substituted with phenyl having piperazinyl substituted with cyclohexyl, benzoyl which has imidazothiadiazolyl substituted with phenyl having piperazinyl substituted with cyclohexyl.

The lipopeptide compound [I], its preparation, its dosage, etc. are disclosed in U.S. Pat. Nos. 5,502,033, 5,376,634, 5,569,946, W096/11210 and W099/40108, the disclosures of which are incorporated herein by reference.

It is known that the granulocyte colony stimulating factor (G-CSF) stimulates the production of white blood cells, and its molecular weight is from about 1.8 million to 2.2 million. And it has been used to treat cancer patients whose white blood cell levels are adversely affected by chemotherapy or radiation. It is also known that various types of G-CSF are present. In the present invention, the G-CSF should not be limited and be considered to mean any compound which has its activity.

For applying the G-CSF to human, it is preferable to apply it by intravenous, intramuscular, pulmonary, oral administration, or insufflation. The lipopeptide compound [I] is preferably administered parenterally, but is not limited to that route, and may also be administered by other routes such as oral, intramuscular or subcutaneous, and may be administered simultaneously, separately, sequentially in combination with the G-CSF.

While the dosage of therapeutically effective amount of the polypeptide compound [I] varies from and also depends upon the age and condition of each individual patient to be treated, in the case of intravenous administration, a daily dose of 0.01-20 mg of the polypeptide compound (I) per kg weight of human being in the case of intramuscular administration, a daily dose of 0.1-20 mg of the polypeptide compound (I) per kg weight of human being, in case of oral administration, a daily dose of 0.5-50 mg of the polypetide compound (I) per kg weight of human being is generally given for treating or preventing infectious diseases.

While the dosage of therapeutically effective amount of the G-CSF varies from and also depends upon the age and condition of each individual patient to be treated, in the case of intravenous administration, a daily dose of 1-100 μg of the G-CSF per kg weight of human being in the case of intramuscular administration, a daily dose of 10-100 μg of the G-CSF per kg weight of human being, in case of oral administration, a daily dose of 5-1000 μg of the G-CSF per kg weight of human being is generally given for treating or preventing infectious diseases.

In more details, the antifungal combination use of the present invention is effective, particularly against the following fungi.

Acremonium;

Absidia (e.g., Absidia corymbifera, etc);

Aspergillus (e.g., Aspergillus clavatus, Aspergillus flavus, Aspergillus fumigatus, Aspergillus nidulans, Aspergillus niger, Aspergillus terreus, Aspergillus versicolor, etc);

Blastomyces (e.g., Blastomyces dermatitidis, etc);

Candida (e.g., Candida albicans, Candida glabrata, Candida guilliermondii, Candida kefyr, Candida krusei, Candida parapsilosis, Candida stellatoidea, Candida tropicalis, Candida utilis, etc.);

Cladosporium (e.g., Cladosporium trichoides, etc);

Coccidioides (e.g., Coccidioides immitis, etc);

Cryptococcus (e.g., Cryptococcus neoformans, etc);

Cunninghamella (e.g., Cunninghamella elegans, etc);

Dermatophyte;

Exophiala (e.g., Exophiala dermatitidis, Exophiala spinifera, etc).

Epidermophyton (e.g., Epidermophyton floccosum, etc);

Fonsecaea (e.g., Fonsecaea pedrosoi, etc);

Fusarium (e.g., Fusarium solani, etc);

Geotrichum (e.g., Geotrichum candiddum, etc);

Histoplasma (e.g., Histoplasma capsulatum var. capsulatum, etc);

Malassezia (e.g., Malassezia furfur, etc);

Microsporum (e.g., Microsporum canis, Microsporum gypseum, etc);

Mucor;

Paracoccidioides (e.g., Paracoccidioides brasiliensis, etc);

Penicillium (e.g., Penicillium marneffei, etc);

Phialophora;

Pneumocystis (e.g., Pneumocystis carinii, etc);

Pseudallescheria (e.g., Pseudallescheria boydii, etc);

Rhizopus (e.g., Rhizopus microsporus var. rhizopodiformis, Rhizopus oryzae, etc);

Saccharomnyces (e.g., Saccharomyces cerevisiae, etc);

Scopulariopsis;

Sporothrix (e.g., Sporothrix schenckii, etc);

Trichophyton (e.g., Trichophyton mentagrophytes, Trichophyton rubruim, etc);

Trichosporon (e.g., Trichosporon asahii, Trichosporon cutaneum, etc).

The above fungi are well known to cause various infection diseases in skin, hair, nail, oral mucosa, gastrointestinal tract, bronchus, lung, endocardium, brain, meninges, urinary organ, vaginal protion, oral cavity, ophthalmus, systemic, kidney, bronchus, heart, external auditory canal, bone, nasal cavity, paranasal cavity, spleen, liver, hypodermal tissue, lymph duct, gastrointestine, articulation, muscle, tendon, interstitial plasma cell in lung, and so on.

Therefore, the combination use of the present invention are useful for preventing and treating various infectious diseases, such as dermatophytosis (e.g., trichophytosis, etc), pityriasis versicolor, candidiasis, cryptococcosis, geotrichosis, trichosporosis, aspergillosis, penicilliosis, fusariosis, zygomycosis, sporotrichosis, chromomycosis, coccidioidomycosis, histoplasmosis, blastomycosis, paracoccidioidomycosis, pseudallescheriosis, mycetoma, mycotic keratitis, otomycosis, pneumocystosis, and so on.

The invention is further described in connection with the following non-limiting example.

### EXAMPLES

Antifungal combination therapy with G-CSF and lipopeptide compound [I] in experimental disseminated Cadidiasis

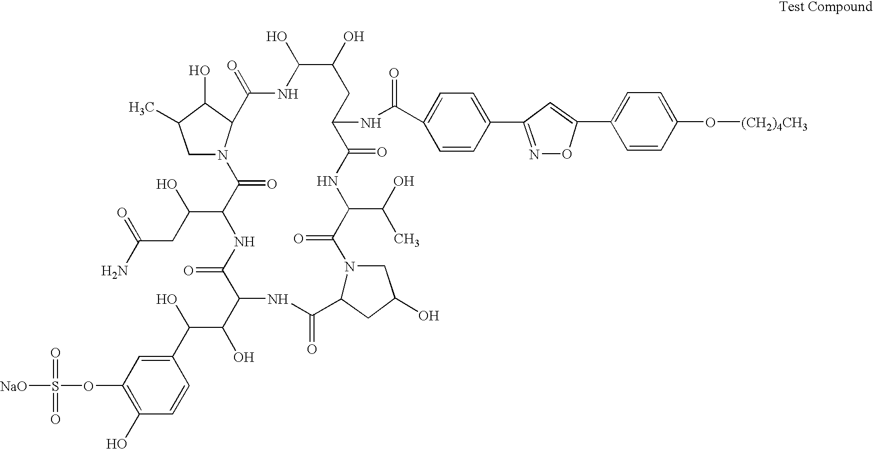

### Test Method

Disseminated candidiasis were induced in ICR mice by the intravenous inoculation of 0.2 ml of cell suspension of Candida albicans FP633 via their lateral tail veins. Cyclophosphamide was administered intraperitoneally at 200 mg/kg 4 days before and 1 day after infection. G-CSF was administered intraperitoneally once daily for 5 days starting at 3 days before infection. Test Compound was administered once daily for 4 days starting at 1 hour after infection by intravenous injection. The survival rate (%) was calculated at 6 days after infection.

### Test Result

1) Survival rate of neutropenic mice infected with C. albicans on day 6 after infection

| | |
|---|---|
| G-CSF | Test Compound (mg/kg) |

| (μg/kg) | 0 | 0.2 |
|---|---|---|
| 0 | 0% | 25% |
| 2.5 | 0% | 62.5% |
| 5 | 25% | 62.5% |
| 10 | 50% | 87.5% |

From the result of the above example, it is confirmed that combination using G-CSF and the lipopeptide compound [I] is effective against fungal infections caused by the fungal pathogens. Accordingly, it is intended that the above examples should be construed as illustrative and that the invention disclosed herein should be limited only by the following claims.

And further, present invention also relates to Commercial package comprising the pharmaceutical composition of the present invention and a written matter associated therewith, wherein the written matter states that the pharmaceutical composition can or should be used for preventing or treating infectious diseases.

1. A method for treatment or inhibition of the infectious diseases caused by the fungal pathogen which comprises administering an effective amount of a lipopeptide compound [I] of the following formula: 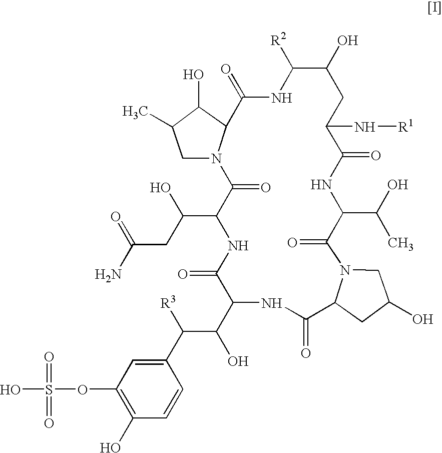
wherein R¹ is acyl group,
R² is hydrogen or hydroxy and
R³ is hydrogen or hydroxy, or a salt thereof, in combination with Granulocyte-colony stimulating factor:

2. The method of claim 1, wherein the lipopeptide compound [I] is 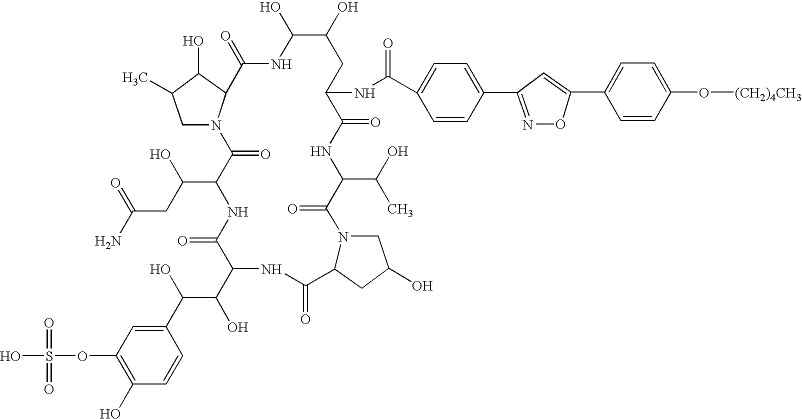
or a salt thereof.

3. The method of claim 1, wherein the infectious diseases are caused by a fungal pathogen selected from Cryptococcus, Candida, Aspergillus, Histoplasma, Coccidioides, Paracoccidioides, Blastomyces, Fusarium, Sporothrix, Trichosporon, Rhizopus, Pseudallescheria, dermatophytes, Paeciliomyces, Alternaria, Curvularia, Exophiala, Wangiella, Penicillium, Saccharomyces, Dematiaceous fungi or Pneumocystis carinii.

4. The method of claim 3, wherein the fungal pathogen is selected from Cryptococcus, Candida or Aspergillus.

5. A pharmaceutical composition for the prophylactic and/or therapeutic treatment of the infectious diseases caused by the fungal pathogen which comprises the lipopeptide compound [I] in claim 1 in combination with G-CSF (Granulocyte-colony stimulating factor) and optionally pharmaceutically carriers or excipients.

6. Use of the lipopeptide compound [I] in claim 1 for the manufacture of medicament for simultaneous, separate or sequential use for the prevention and/or treatment of the infectious diseases caused by the fungal pathogen in combination with Granulocyte-colony stimulating factor.

7. A commercial package comprising the pharmaceutical composition of claim 5 and a written matter associated therewith, wherein the written matter states that the pharmaceutical composition can or should be used for preventing or treating infectious diseases.
